(19) United States
(12) Patent Application Publication
(10) Pub. No.: US 2009/0297508 A1
(43) Pub. Date: Dec. 3, 2009

(54) Compositions and methods for treating hemostasis disorders associated with clec-2 signal transduction

(75) Inventors: Yukio Ozaki, Chuo-shi (JP); Katsue Inoue, Chuo-shi (JP); Steve P. Watson, Birmingham (GB)

Correspondence Address: BIRCH STEWART KOLASCH & BIRCH, PO BOX 747, FALLS CHURCH, VA, 22040-0747, US

(73) Assignee: UNIVERSITY OF YAMANASHI, Kofu-shi (JP)

(21) Appl. No.: 11/919,117

(22) PCT Filed: Apr. 24, 2006
(86) PCT No.: PCT/JP2006/309066

§ 371 Date: Oct. 24, 2007

(30) Foreign Application Priority Data
Apr. 25, 2005 (US) 60/674287

(51) Int. Cl. A61K 39/395; G01N 33/53; A61K 38/00; A61K 9/00; A61K 31/7088; G01N 33/566; G01N 33/567

(52) U.S. Cl. 424/133.1; 435/7.21; 436/501; 424/172.1; 514/12; 514/44 R; 800/14

(57) ABSTRACT

The present invention provides a method for screening compounds to identify therapeutic candidates for treating hemostasis disorders, the method includes the steps of contacting a cell that expresses a CLEC-2 receptor with a test compound and a CLEC-2 receptor ligand, under conditions which, but for the presence of the test compound, permit binding of the CLEC-2 receptor to the CLEC-2 receptor ligand; and detecting if any increase or decrease in an indicator of CLEC-2 receptor activity is observed in comparison to a control; wherein an increase or decrease in the indicator in comparison to the control indicates that the test compound is a candidate for modulating hemostasis.

### TECHNICAL FIELD

The present invention is in the field of signal transduction. More particularly, the present invention relates to compositions and methods to screen for compounds, which modulate platelet activation via a CLEC-2 receptor.

### BACKGROUND ART

Platelets are small anuclear blood cells essential to hemostatic control and wound healing. Although, circulating platelets are fairly quiescent under normal conditions, these cells become activated when a blood vessel is torn or damaged. Platelet activation is mediated by exposure to factors, such as collagen, which bind to receptors on the surface of platelets; this binding instigates signal transduction pathways leading to platelet aggregation, blood coagulation and clot formation.

Clot formation is a major etiologic factor in a wide range of human diseases, most commonly, vascular diseases. Excessive platelet aggregation in arteries and veins contributes to atherosclerotic and arteriosclerotic plaques, which reduce the flow of blood to sensitive tissues. Ultimately, this platelet-dependent buildup may manifest as acute myocardial infarction, chronic unstable angina, transient ischemia, stroke, peripheral vascular diseases, arterial thrombosis, preeclampsia, pulmonary embolism, and restenosis.

Agents that block receptor signaling and mitigate platelet aggregation have been developed to treat clot formation and related diseases. One such class of surface receptors that has been targeted includes the integrin αIIbβ3 receptors. These receptors bind fibrinogen and mediate platelet aggregation. Three classes of integrin αIIbβ3 inhibitors approved for clinical use include: (1) humanized murine monoclonal antibodies directed against the active conformation of integrin αIIbβ3; (2) RGD (Arg-Gly-Asp)-containing peptides that block fibrinogen binding and (3) disintegrin mimicking compounds that compete with fibrinogen binding. Although these inhibitors are effective in reducing platelet aggregation, all three of these classes have a narrow margin of safety.

GPVI is another platelet surface receptor that, likewise, has been used as a target for anti-thrombotic agents. Compounds obtained from the saliva of insects and invertebrates that feed on the blood of higher animals have been shown to impair the contact between collagen, a GPVI ligand, and GPVI surface receptors. Although these agents result in an anti-thrombotic effect, they also have been associated with a high risk of bleeding.

Hence, there is a need in the art to identify additional platelet surface receptors that may be used as targets for developing agents useful in treating diseases relating to platelet aggregation. Additionally, there is a need in the art for methods enabling the identification of agents useful for inhibiting novel platelet surface receptors and their signal transduction pathways. Furthermore, there is a need in the art for compositions that can be used to both mediate the platelet aggregation associated with thrombosis as well as a composition that may be effective in treating excessive bleeding associated with aberrant platelet surface receptor function.

### DISCLOSURE OF INVENTION

The present invention provides a method for screening compounds to identify therapeutic candidates for treating hemostasis disorders, the method includes the steps of contacting a cell that expresses a CLEC-2 receptor with a test compound and optionally, a CLEC-2 receptor ligand, under conditions which, but for the presence of the test compound, permit binding of the CLEC-2 receptor to the CLEC-2 receptor ligand; and detecting whether the compound increases or decreases an indicator of CLEC-2 receptor activity in comparison to a control; wherein an increase or decrease in the indicator in comparison to the control indicates that the test compound is a candidate for modulating hemostasis.

Furthermore, the present invention provides a method for screening compounds to identify therapeutic candidates for treating hemostasis disorders including the steps of a) contacting a cell that expresses a CLEC-2 receptor with a test compound; and b) detecting any change in an indicator of CLEC-2 receptor activity in comparison to a control.

Additionally, the present invention provides a method for screening compounds to identify therapeutic candidates for modulating hemostasis, the method includes the steps of administering a test compound and optionally, a CLEC-2 receptor ligand to a non-human animal that expresses a CLEC-2 receptor; and detecting whether the compound increases or decreases an indicator of CLEC-2 receptor activity in said animal in comparison to a control animal; wherein a decrease in the indicator indicates that the compound is a candidate for decreasing cellular aggregation by decreasing CLEC-2 receptor activity in vivo; and wherein an increase in the indicator indicates that the compound is a candidate for increasing cellular aggregation by increasing CLEC-2 receptor activity in vivo.

Furthermore, the present invention provides a method for screening compounds to identify therapeutic candidates for modulating hemostasis, the method includes the steps of contacting a compound and a CLEC-2 receptor polypeptide or fragment thereof; and

detecting binding between the CLEC-2 receptor polypeptide and the compound.

Also, the present invention provides a method for screening compounds to identify therapeutic candidates for treating hemostasis disorders, including the steps of contacting a CLEC-2 polypeptide having a phosphorylated tyrosine in a cytoplasmic domain, or contacting a fragment thereof comprising the phosphorylated cytoplasmic domain, with a test compound; and detecting whether the compound modulates binding of the signaling partner to a CLEC-2 polypeptide; wherein the signaling partner is a member of the Syk family of tyrosine kinases.

Furthermore, the present invention provides a method for reducing the severity of a pathological state mediated by CLEC-2 signaling including the steps of contacting a CLEC-2 receptor having a phosphorylated tyrosine in a cytoplasmic domain, or contacting a fragment thereof comprising the phosphorylated cytoplasmic domain, with a compound which blocks the binding of the signaling partner to the CLEC-2 receptor.

Additionally, the present invention provides a method for reducing the severity of a pathological state mediated by CLEC-2 signaling including the step of administering to a mammal an antagonist of CLEC-2 signaling.

Furthermore, the present invention provides a method for reducing the severity of a pathological state mediated by a lack of CLEC-2 signaling including the step of administering to a mammal an agonist of CLEC-2 signaling.

Additionally, the present invention provides a CLEC-2 antagonist including an isolated antibody, which specifically binds to a polypeptide of SEQ ID NO: 2, or fragments thereof.

Furthermore, the present invention provides a pharmaceutical composition including a polypeptide of SEQ ID NO: 2, or a fragment thereof, or a salt thereof, and a pharmaceutical carrier.

Finally, the present invention provides a pharmaceutical composition including a nucleic acid encoding SEQ ID NO: 2, or a fragment thereof or a salt thereof and a pharmaceutical carrier.

### BRIEF DESCRIPTION OF DRAWINGS

FIG. 1 Association of CLEC-2 with rhodocytin-coated beads.

A) Washed platelets were labeled with biotin and lysed with an equal volume of 2× lysis buffer. They were precleared, and incubated with rhodocytin-bound (rhod-Seph) or glycin-bound Sepharose 4B (gly-Seph) for 4 hours at 4° C. After extensive washing, proteins were eluted from the beads with SDS reducing sample buffer. Precipitated platelet proteins were separated by 4-20% SDS-PAGE and detected by horse radish peroxidase-conjugated streptavidin (avidin-HRP). This is representative of two experiments. B) Pull down and electrophoresis were performed as described in (A) using unlabeled washed platelets. Ligand blotting was performed by biotin-conjugated rhodocytin (rhodocytin-biotin) and avidin-HRP. C) Compiled Data Output File for Mascot MS Searches Swiss-prot Search results. D) Washed human platelets were incubated with control goat IgG or anti-CLEC-2 antibody, followed by staining with FITC-conjugated anti-goat IgG. Samples were analyzed with a Becton Dickinson FACScan. E) Washed platelets were dissolved in 4×SDS sample buffer, separated by SDS-PAGE, and blotted with anti-CLEC-2 antibody (left panel) or control goat IgG (right panel). The data is representative of 5 experiments. F) The membrane used in (B) was reprobed with anti-CLEC-2 antibody. G) Densitometric analysis of 32 and 40 kDa CLEC-2 bands in (E) was performed by Molecular Imager FX and Quantity One software (BIO-RAD). Relative intensity of the 40 kDa band compared with 32 kDa band was expressed as mean±S.E. (n=4).

FIG. 2 Selective response of CLEC-2 expressing 293T-REx™ cells to rhodocytin.

293T-REx™ cells, that express CLEC-2 under a tet repressor, were incubated with vehicle or 1 μg/ml of doxycycline for 24-48 hours. 5×10⁶ cells were incubated with an antibody to CLEC-2 or an isotype matched control and analyzed by FACscan. B) 293T-REx™ cells (5×10⁶/ml) pretreated with or without doxycycline were preincubated with 100 nM rhodocytin. After excess of rhodocytin was removed by centrifugation, cells were incubated with control rabbit IgG or anti-rhodocytin antibody, followed by FITC-conjugated anti-rabbit IgG. C) Cells were stimulated with or without 500 nM rhodocytin for 10 min, dissolved with SDS sample buffer, separated by SDS-PAGE. Protein tyrosine phosphorylation was detected by western blotting with anti-phospho tyrosine antibody (4G10). D) 1×10⁷ cells were dissolved with 4×SDS sample buffer. Proteins were separated by SDS-PAGE and blotted with anti-CLEC-2 antibody (left panel) or control-goat IgG (right panel). The data are representative of two to three experiments.

FIG. 3 Platelet aggregation and CLEC-2 tyrosine phosphorylation induced by cross-linking of CLEC-2 by anti-CLEC-2 antibody.

A) Human washed platelets (2×10⁸/ml) were stimulated by 10 μg/ml of anti-CLEC-2 antibody or control goat IgG in the presence of F(ab)′₂ fragment of anti-FcγRIIA antibody (IV.3). Platelet aggregation was monitored with an aggregometer. B) Human washed platelets (1×10⁹/ml) were lysed with 2× lysis buffer after stimulation as described above. CLEC-2 was immunoprecipitated and sequentially western blotted with anti-phosphotyrosine antibody (4G10) or monoclonal anti-CLEC-2 antibody. The data are representative of four experiments.

FIG. 4 Tyrosine phosphorylation of CLEC-2 and signaling molecules downstream GPVI upon rhodocytin stimulation.

A) Human washed platelets (2×10⁸/ml) were stimulated by 300 nM rhodocytin and platelet aggregation was monitored using an aggregometer. B-E) Washed human platelets (300 or 500 μl at 1×10⁹/ml) were stimulated with 300 nM (B) rhodocytin, 50 nM (C-E) rhodocytin, 50 mg/ml of collagen, or 100 μM SFLLRN (SEQ ID NO: 6) for the indicated times. In D), platelets were pretreated with 30 μM PP3 or PP2, prior to stimulation with rhodocytin. Reactions were terminated by addition of an equal volume of 2× lysis buffer. Platelet lysates were precleared and detergent-insoluble debris was removed by centrifugation. Antibodies against CLEC-2 (B), PLCγ2, Syk, Btk, SLP-76, or LAT (E) were added to the resultant supernatant and incubated overnight with protein A or G Sepharose. Precipitated proteins were separated by SDS-PAGE and western blotted with the indicated antibodies. The data are representative of 2-5 experiments.

FIG. 5 Crucial role of Syk in platelet activation downstream of CLEC-2.

(A) Washed murine platelets were stimulated with 50 nM rhodocytin for the indicated times. Whole cell lysates or immunoprecipitates with antibodies against PLCγ2, Vav1, or Vav3 were separated by SDS-PAGE and western blotted with the indicated antibodies. (B) Control or Syk-deficient platelets were stimulated with 30 nM rhodocytin and platelet aggregation was monitored using an aggregometer. (C) (i)(ii) Washed human platelets, pretreated with or without 30 μM PP2, were stimulated with 50 nM rhodocytin or 10 μg/ml of convulxin for the indicated times. Reactions were terminated by addition of an equal volume of 2× lysis buffer. Platelet lysates were precleared and detergent-insoluble debris was clarified by centrifugation. The resultant supernatant was incubated with 40 μl of glutathione beads associated with GST fusion protein containing tandem Syk SH2 domains. Precipitated proteins were separated by SDS-PAGE and western blotted with an antibody to CLEC-2. (iii) CLEC-2 associated with 10 μg of CLEC-2 phospho-YXXL-containing peptide plus avidin-sepharose was detected by CLEC-2 antibody. The data are representative of 2-5 experiments.

FIG. 6 Inhibition of rhodocytin-induced platelet aggregation in mice deficient in PLCγ2, LAT, SLP-76, or Vav1/3.

Murine washed platelets from wild-type mice or mice deficient in PLCγ2, LAT, SLP-76, or Vav1/3 were stimulated with indicated concentrations of rhodocytin and platelet aggregation was monitored using an aggregometer. Results are representative of 3-6 experiments.

### BEST MODE FOR CARRYING OUT THE INVENTION

The inventors have surprisingly identified that the C-type lectin receptor, CLEC-2, activates platelets upon response to rhodocytin, a snake venom, or a CLEC-2 antibody. As described in the Examples, the inventors used rhodocytin affinity chromatography and mass spectrometry in an attempt to identify one or more receptors that could underlie platelet activation by rhodocytin. This approach led to the realization that the receptor CLEC-2, which displays a single carbohydrate recognition domain and a cytoplasmic tyrosine-based motif, is able to confer signaling responses to rhodocytin when expressed in a cell line. Significantly, CLEC-2 has a single tyrosine in its cytosolic tail that represents one-half of an immunoreceptor tyrosine-based activation motif (ITAM), YXXL, which undergoes tyrosine phosphorylation upon activation by rhodocytin downstream of Src kinases. Furthermore, tyrosine-phosphorylated CLEC-2 is precipitated by the tandem SH2 domains of Syk, a member of the Syk family of tyrosine kinases, and activation of platelets by rhodocytin is abolished in the absence of Syk. These results demonstrate that CLEC-2 is a platelet activation receptor that underlies activation by the snake toxin rhodocytin.

Based on these observations, the invention provides methods and compositions for the treatment of hemostasis disorders attributed to cellular activation or a lack of cellular activation. Preferably the cells are platelets. Cellular activation includes, but is not limited to, a change in cell shape or cellular aggregation.

As used herein “hemostasis” refers to effective, appropriate, stoppage of bleeding or hemorrhage. “Hemostasis disorders” as used herein, refers to conditions or diseases including, but not limited to, excessive bleeding and abnormal blood clotting. Abnormal blood clotting is associated with diseases including, but not limited to, acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

### Inventive Assays of the Invention

The present invention herein provides methods for screening compounds to identify therapeutic candidates for treating hemostasis disorders using cell-based and cell-free assays.

### Cell-Based Assays of the Invention

In some embodiments, the inventive methods employ cells to screen for compounds to identify therapeutic candidates for treating hemostasis disorders.

In one embodiment, the inventive method includes a) contacting a cell that expresses a CLEC-2 receptor with a test compound and optionally, a CLEC-2 receptor ligand under conditions which, but for the presence of the compound, permit binding of the CLEC-2 receptor to the CLEC-2 receptor ligand; and b) detecting if any increase or decrease in an indicator of CLEC-2 receptor activity is observed in comparison to a control. An increase or decrease in the indicator signifies that the compound is a candidate for modulating hemostasis by interacting with a CLEC-2 receptor.

In this embodiment, a test compound is contacted with cells expressing the CLEC-2 receptor on the cells' surface. The contacting step is conducted under conditions which, but for the presence of the compound, would permit the binding of the CLEC-2 receptor to a CLEC-2 receptor ligand. Thus, for example, the contacting step can be carried out in a physiologically acceptable buffer solution or in the cell culture medium in which the cells are grown. Preferably, the conditions are chosen to be substantially duplicative of in vivo conditions.

As used herein a “CLEC-2 receptor ligand” refers to a molecule that binds to the binding domain of a CLEC-2 receptor that is expressed on the surface of cells.

In one embodiment, the CLEC-2 receptor ligand is an antagonist of CLEC-2 receptor activity.

In another embodiment, the CLEC-2 receptor ligand is an agonist of CLEC-2 receptor activity. Preferably, the agonist ligand is a CLEC-2 antibody, such as the CLEC-2 antibody from R & D Systems (Minneapolis, Minn.). Even more preferably, the CLEC-2 agonist ligand is rhodocytin.

The indicators of CLEC-2 receptor activity are based on the indicators observed when platelet cells are contacted with the agonist, rhodocytin, or a CLEC-2 antibody (See, Examples). Upon contact with a receptor ligand agonist, the CLEC-2 receptor will activate a signaling cascade that results in indicators, including but not limited to, 1) the CLEC-2 receptor is phosphorylated, more preferably, the YXXL motif in the cytoplasmic domain of CLEC-2 is phosphorylated; 2) a member of the Syk family of tyrosine kinases interacts with the cytoplasmic domain of CLEC-2, preferably the member is Syk; and 3) cellular activation occurs.

In one embodiment, cellular activation is indicated by a change in cell shape when compared to the shape of cells in their non-activated or quiescent shape. Preferably, the cells are platelets.

In another embodiment, cellular activation is indicated by cellular aggregation.

Preferably, the cellular aggregation is platelet aggregation.

Methods of measuring indicators of CLEC-2 activity are described in the Examples below. Cellular activation can be assessed according to any method known in the art, including that described in Example 1(h). Tyrosine phosphorylation of CLEC-2 also can be assessed according to any method known in the art, including in the manner described Example 5. Syk interaction with the cytoplasmic tail of CLEC-2 can be assessed indirectly according to the method described in Example 6.

The presence or absence or the amount of these indicators are compared to those of control cells, optionally which have been contacted with a CLEC-2 ligand, but not with the test compound. Those test compounds which decrease the amount or presence of indicators in comparison to control cells, are antagonists of CLEC-2 receptor signaling and can be used as candidate compounds for treating those hemostasis disorders associated with blood clotting, such as cellular aggregation, preferably, platelet aggregation. Alternatively, test compounds resulting in an increase in the amount or presence of indictors in comparison to control cells, can be used as candidate compounds for treating those hemostasis disorders associated with excessive bleeding, such as decreased or absent cellular aggregation, preferably, decreased or absent platelet aggregation.

In another embodiment, the present invention provides a method for screening compounds to identify therapeutic candidates for treating hemostasis disorders including the steps of a) contacting a cell that expresses a CLEC-2 receptor with a test compound; and b) detecting any change in an indicator of CLEC-2 receptor activity in comparison to a control. A change in the indicator signifies that the compound is a candidate for modulating hemostasis by interacting with a CLEC-2 receptor.

Preferably, the cells are initially activated with a CLEC-2 receptor ligand. In one embodiment, the CLEC-2 receptor ligand is an agonist. In another embodiment, the CLEC-2 receptor ligand is an antagonist.

Preferably, the inventive methods for screening compounds to identify therapeutic candidates for modulating hemostasis that utilize living cells, which express the CLEC-2 receptor, employ cells that naturally express the CLEC-2 receptor. In another embodiment, the inventive methods employ cells that have been genetically engineered to cause or enhance CLEC-2 receptor expression.

In yet another preferred embodiment, when the cells are transformed with a genetic construct, sequences that encode a chimeric protein are used. The chimeric protein encodes for the binding domain and the cytoplasmic domain including the YXXL motif, and functions substantially similar to a natural CLEC-2 receptor.

The cells of the instant invention are preferably mammalian (e.g., human, monkey, mouse, rat, canine, hamster, rabbit, goat), or can be other eukaryotic (e.g., insect or yeast) or prokaryotic (e.g., bacterial) cells, which have been genetically engineered to express a human or other mammalian CLEC-2 receptor. The cells can be obtained from immortalized cell lines, cell cultures, or primary cell preparations, or can be obtained directly from animals (e.g., a human or other mammal, or a non-human transgenic animal).

In more preferred embodiments, the cells are human, mouse, rat, or hamster cells expressing the human CLEC-2 receptor.

In even more preferred embodiments, the cells are human or murine and express the human CLEC-2 receptor.

In other preferred embodiments, the cells are platelets. In an even more preferred embodiment, the cells are platelets, which express the human CLEC-2 receptor endogenously.

### Genetic Constructs

To produce the transformed cells and/or transgenic non-human animals (see below) of the present invention, a variety of standard recombinant genetic constructs can be employed.

The genetic constructs are introduced into target cells as structural components of any of a wide range of vectors that can be specifically or nonspecifically inserted into the target cell genome. Suitable expression vectors include bacterial, plasmid, yeast, and viral vectors. The viral vectors include, but are not limited to, herpes simplex virus vectors, adenovirus vectors, adeno-associated virus vectors, retroviral vectors, lentiviral vectors, pseudorabies virus vectors, alpha-herpes virus vectors, and the like. A thorough review of viral vectors, and how to use such vectors in conjunction with the expression of polynucleotides of interest can be found in Viral Vectors: Gene Therapy and Neuroscience Applications, Caplitt and Loewy, eds., Academic Press, San Diego, 1995.

Retroviral vectors can be used in conjunction with retroviral packaging cell lines, such as those described in U.S. Pat. No. 5,449,614. Where non-murine mammalian cells are used as target cells for genetic modification, amphotropic or pantropic packaging cell lines can be used to package suitable vectors (Ory et al. (1996), Proc. Natl. Acad. Sci. (USA) 93:11400-11406). Representative retroviral vectors are described, for example, in U.S. Pat. No. 5,521,076.

Genetic constructs intended to cause or enhance expression of the CLEC-2 receptor and/or fragments thereof will typically include genetic sequences encoding at least the cytoplasmic domain including the YXXL motif, operably linked to transcriptional control elements which are exogenous to the CLEC-2 sequences (and possibly exogenous to the genome of the target cell). These transcriptional control elements will include a transcriptional promoter region and, optionally, enhancer sequences. Examples of transcriptional promoters and enhancers that are incorporated into a construct include, but are not limited to, cell- or tissue-specific promoters, inducible promoters, and regulatable promoters. Specific examples include, but are not limited to, the herpes simplex thymidine kinase promoter, the cytomegalovirus (CMV) promoter/enhancer, SV40 promoters, PGK promoter, metallothionein promoter, adenovirus late promoter, vaccinia virus 7.5K promoter, avian beta globin promoter, histone promoters (e.g., mouse histone H3-614 promoter), beta actin promoter, and the cauliflower mosaic virus 35S promoter (see generally, Sambrook et al., Molecular Cloning, Vols. I-III, Cold Spring Harbor Laboratory Press, Cold Spring Harbor, N.Y. (1989), and Current Protocols in Molecular Biology, John Wiley & Sons (1989-2000 editions)).

The genetic constructs described above utilize nucleic acids that encode for CLEC-2 polypeptide sequences as described below. As used herein, the terms peptide and polypeptide are used interchangeably.

For purposes of this invention, “nucleic acid” is defined as RNA or DNA that encodes a polypeptide that includes SEQ ID NO: 2, biological fragments thereof, as defined below, or those having sequence identity to SEQ ID NO: 2 or biological fragments thereof, or is complementary to a nucleic acid sequence encoding such polypeptides, or hybridizes to such nucleic acids and remains stably bound to it under stringent conditions. Specifically contemplated are genomic DNA, cDNA, mRNA, antisense molecules, as well as siRNAs.

Homology or identity at the nucleotide or amino acid sequence level is determined by BLAST (Basic Local Alignment Search Tool) analysis using the algorithm employed by the programs blastp, blastn, blastx, tblastn and tblastx (Karlin et al, Proc Nad Acad Sci USA (1990) 87: 2264-2268 and Altschul, S. F., J. Mol. Evol. (1993) 36: 290-300, herein incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a pre-selected threshold of significance. For a discussion of basic issues in similarity searching of sequence databases, see Altschul et al. (Nature Genetics (1994) 6:119-129), which is herein incorporated by reference. The search parameters for histogram, descriptions, alignments, expect (i.e., the statistical significance threshold for reporting matches against database sequences), cutoff, matrix and filter are at the default settings.

The default scoring matrix used by blastp, blastx, tblastn, and tblastx is the BLOSUM62 matrix (Henikoff et al., Proc Natl, Acad Sci USA (1992) 89: 10915-10919, fully incorporated by reference). For blastn, the scoring matrix is set by the ratios of M (i.e., the reward score for a pair of matching residues) to N (i.e., the penalty score for mismatching residues), wherein the default values for M and N are −5 and −4, respectively.

In another embodiment, the nucleic acid sequences employed in the present invention are those that hybridize to the disclosed sequences under stringent conditions. As used herein, “stringent conditions” are those that (1) employ low ionic strength and high temperature for washing, for example, 0.015M NaCl/0.0015M sodium titrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

Preferably, the nucleic acids of the instant invention encode for SEQ ID NO: 2 and biological fragments thereof. Preferred fragments are those that encode the CLEC-2 cytoplasmic domain having the YXXL motif, which can be phosphorylated by a tyrosine kinase.

Additionally, nucleic acids that encode homologous portions of allelic variants of SEQ ID NO: 2 and fragments thereof, as well as conservative amino acid substitutions of SEQ ID NO: 2 and fragments thereof, are contemplated as well. As used herein, an allelic variant refers to a naturally occurring CLEC-2 having a different amino acid sequence than that specifically recited herein. Allelic variants, though possessing a different amino acid sequence that those recited above, will still have the requisite binding domain expressed on the surface of cells and/or the phosphorylatable tyrosine residue recognized by a CLEC-2 signaling partner, and will function to associate or interact with this partner as part of the relevant signaling cascade.

As used herein, a conservative amino acid substitution refers to alterations in the amino acid sequence that do not adversely effect a peptide. A substitution is said to adversely effect the peptide when the altered sequence prevents a CLEC-2 ligand from binding to the binding domain of the CLEC-2 receptor when expressed on the cell surface and/or the phosphorylation of the peptide or the ability of the phosphorylated peptide to associate with a signaling partner is prevented. For example, the overall charge, structure or hydrophobic/hydrophilic properties of the peptide can be altered without adversely effecting the peptide. Accordingly, the amino acid sequence of the above peptides can be altered, for example to render the peptide more hydrophobic or hydrophilic, without adversely effecting the ability of the peptide to bind to a ligand and/or to become phosphorylated and/or the ability of the phosphorylated peptide to associate with a signaling partner.

Ordinarily, the nucleic acids for use in the instant invention encode for peptides and analogs including an amino acid sequence having at least 75% amino acid sequence identity with the disclosed peptides from the CLEC-2 receptor, more preferably at least 80%, even more preferably at least 90%, and most preferably at least 95%. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity.

Thus, the nucleic acids encoding peptides and analog molecules that are useful in the instant invention include molecules encoding peptides having the peptide disclosed herein; fragments thereof having a consecutive sequence of at least about 3, 4, 5, 10 or 15 amino acid residues from the corresponding CLEC-2 cytoplasmic domain including the YXXL motif; amino acid sequence variants of the disclosed sequences or their fragments as defined above, which have been substituted by another residue. Contemplated nucleic acids also encode polypeptides that include those containing predetermined mutations by, e.g., homologous recombination, site-directed or PCR mutagenesis, and the corresponding CLEC-2 receptor or their fragments of other animal species, including but not limited to rabbit, rat, murine, porcine, bovine, ovine, equine and non-human primate species, and the alleles or other naturally occurring variants of the CLEC-2 cytoplasmic domain of the foregoing species and of human sequences; derivatives wherein the peptides or their fragments have been covalently modified, by chemical, enzymatic, or other appropriate means, to attach a moiety other than a naturally occurring amino acid (for example a detectable moiety such as an enzyme or radioisotope); glycosylation variants (insertion of a glycosylation site or deletion of any glycosylation site by deletion, insertion or substitution of appropriate amino acid); and soluble forms as well as irreversibly phosphorylated forms.

### Animal-Based Assays

In another aspect, the present invention provides methods for screening compounds to identify therapeutic candidates for modulating hemostasis, in which whole non-human animals are employed. Thus, in this aspect, a test compound is administered to an animal along with a CLEC-2 receptor ligand as described herein above, and an increase or decrease in an indicator of CLEC-2 receptor activity is detected in the animal in comparison to a control animal.

In one embodiment, the control animal is contacted with a CLEC-2 receptor ligand, but not a test compound.

An increase in an indicator, as described above, in comparison to the control animal, signifies that the compound is a candidate useful for increasing cellular aggregation by increasing CLEC-2 receptor activity in vivo. Preferably, a decrease in an indicator is seen in comparison to a control, which signifies that the compound is a candidate for decreasing cellular aggregation by decreasing CLEC-2 receptor activity in vivo. Preferably, the cellular aggregation is platelet aggregation.

In some embodiments, the animal is an animal which normally expresses the CLEC-2 receptor and which is normal or wild-type, with respect to the receptor. Alternatively, the animal is a transgenic non-human animal, or a descendant of such an animal, which has been transformed with a genetic construct encoding a CLEC-2 receptor, which is expressed in the animal, preferably in tissues that express an endogenous CLEC-2 receptor. Transformation vectors can be produced and used to transform blastocysts or embryonic stem cells, and these transformed cells can then be used to produce transgenic animals by methods known in the art (see, e.g., Solle et al. (2001), J. Biol. Chem. 276:125-132, herein incorporated by reference).

### Assays for Modulators of CLEC-2 Expression

In another aspect, the present invention provides methods for screening compounds, which may modulate the expression of a CLEC-2 receptor by modulating the transcription of a CLEC-2 receptor gene. Such compounds are therapeutic candidates for modulating hemostasis disorders.

Preferably, cells that express an endogenous CLEC-2 receptor are used, and expression of the sequence encoding the CLEC-2 receptor is measured by detecting CLEC-2 mRNA or protein levels using standard methods known in the art.

### Cell-Free Binding Assays

Also provided herein are methods for screening for compounds, which bind to the CLEC-2 receptor in a cell-free assay. In one embodiment, the invention provides a method for screening compounds to identify therapeutic candidates for treating hemostasis disorders by a) contacting a compound with a CLEC-2 receptor polypeptide or fragment thereof and detecting binding of the CLEC-2 polypeptide with the test compound, wherein binding between the CLEC-2 receptor polypeptide and the test compound indicates that the compound is a candidate for modulating hemostasis.

Preferably polypeptides or nucleic acids encoding SEQ ID NO: 2 and fragments thereof are used in the instant methods. More preferably, nucleic acids encoding a CLEC-2 fragment of SEQ ID NO: 3 are used. In yet another preferred embodiment, the CLEC-2 polypeptide includes at least the YXXL motif.

In yet another embodiment, the instant invention provides a method for screening compounds to identify therapeutic candidates for treating hemostasis disorders by contacting a CLEC-2 polypeptide having a phosphorylated tyrosine in a cytoplasmic domain, or contacting a fragment thereof comprising the phosphorylated tyrosine, with a test compound and detecting whether the compound modulates the binding of a signaling partner to the CLEC-2 polypeptide, wherein the signaling partner is a member of the Syk family of tyrosine kinases.

In this embodiment, a CLEC-2 polypeptide or fragment thereof, including at least the phosphorylated YXXL motif, is employed. The CLEC-2 polypeptide or fragment thereof can be phosphorylated by any method known in the art. The CLEC-2 polypeptide or fragment thereof is incubated with an identified signaling partner in the presence and absence of a compound to be tested.

Preferably, the signaling partner is a member of the Syk family of tyrosine kinases. More preferably, the signaling partner is Syk.

As used herein a “signaling partner” refers to a molecule, which, through interaction with a CLEC-2 polypeptide or fragment thereof, modulates transduction of signals across cell membranes. For example, members of the Syk-family of tyrosine kinases would be embraced by the term.

After incubation, under conditions that allow association of a CLEC-2 polypeptide or fragments thereof with a signaling partner, the two compositions are analyzed and compared to determine if the test compound is capable of modulating the association of the CLEC-2 polypeptide or fragments thereof with the signaling partner.

In one embodiment, the test compound blocks the binding of the CLEC-2 polypeptide or fragments thereof to the signaling partner by selectively binding to the phosphorylated cytoplasmic domain of the CLEC-2 receptor polypeptide or fragments thereof.

In another embodiment, the test compound blocks the binding of the CLEC-2 receptor polypeptide or fragments thereof, by selectively binding to the signaling partner.

In another embodiment, the test compound blocks the interaction between the CLEC-2 polypeptide or fragments thereof and reduces cellular aggregation of a CLEC-2 expressing cell. Preferably, the cellular aggregation is platelet aggregation.

In any of the preceding embodiments, in order to facilitate detection of binding amongst the compound and the CLEC-2 receptor polypeptide or fragments thereof; or the compound and the signaling partner, or the signaling partner and the CLEC-2 receptor polypeptide, either the compound or the signaling partner are preferably labeled with a detectable label. After contacting the labeled element with the CLEC-2 polypeptide or fragment thereof, any unbound labeled element is washed away, and the labeled element bound to the CLEC-2 polypeptide or fragment thereof is detected.

Labels which can be used in the foregoing binding assays include, but are not limited to, radioisotopes (e.g., ³²P, ³⁵S), fluorescent labels (e.g., fluorescein isothiocyanate, rhodamine, phycoerythrin, phycocyanin, allophycocyanin, and fluorescamine), chemiluminescent labels (e.g., luciferin, luminol, isoluminol, aromatic acridinium esters, imidazoles, and the oxalate esters), enzymatic domains (e.g., luciferase, alkaline phosphatase, β-galactosidase, glucose-6-phosphate dehydrogenase, maleate dehydrogenase, glucose oxidase, and peroxidase), specific binding elements (e.g., c-myc epitope, biotin or (strept)avidin, Protein A, lectin, immunoglobulins), and metal atoms (e.g., Au).

In preferred embodiments of the foregoing binding assays, one of the non-labeled binding elements is immobilized. Thus, in some embodiments, the compound is labeled and the CLEC-2 polypeptide or fragment thereof is immobilized on a substrate. In other embodiments, the signaling partner is labeled and the CLEC-2 polypeptide is immobilized on a substrate.

Substrates appropriate for immobilizing these elements include affinity columns, slides, wells, particles and/or microparticles.

In another aspect, methods are provided for screening compounds to identify therapeutic candidates for modulating hemostasis, in which aggregation assays are employed. In these methods, a compound is immobilized on a first particle and a CLEC-2 polypeptide or fragment thereof is immobilized on a second particle.

In other embodiments, a compound is immobilized on a first particle and CLEC-2 polypeptide is immobilized on a second particle and the signaling partner is immobilized on a third particle. In embodiments including a signaling partner, the particles are contacted under conditions, which allow binding of the CLEC-2 polypeptide or fragment thereof to a signaling partner unless prevented by a test compound.

Binding between the components is detected by detecting aggregation of the particles. Aggregation indicates that the compound is a candidate for modulating hemostasis by e.g. interacting with a CLEC-2 polypeptide or fragment thereof, or a CLEC-2 polypeptide signaling partner. Aggregation is measured using standard techniques, including turbidity measurements. Particles suitable for use in the invention include latex, polylactic acid (PLA), polyglycolic acid (PGA), poly(co-lactic-glycolic) acid (PLGA), poly(ethylene glycol) (PEG), and similar microspheres, having size ranges of from 1 μm-1000 μm.

### Test Compounds

Examples of classes of compounds that can be screened in all of the preceding embodiments in order to identify compounds that are CLEC-2 receptor agonists or antagonists, and, therefore, can modulate hemostasis include, but are not limited to, nucleic acids (e.g., DNA and RNA), carbohydrates, lipids, proteins, peptides, peptidomimetics, and small molecules.

Test compounds can be identified from libraries of agents that are obtained using any of numerous approaches known in the art, including both libraries of naturally-occurring compounds and libraries of synthetic compounds produced by combinatorial chemistry.

Examples of methods for the synthesis of molecular libraries can be found in the art, for example in: U.S. Pat. No. 5,738,996; U.S. Pat. No. 5,807,683; DeWitt et al. (1993), Proc. Natl. Acad. Sci. (USA) 90:6909; Erb et al. (1994), Proc. Natl. Acad. Sci. (USA) 91:11422; Zuckermann et al. (1994), J. Med. Chem. 37:2678; Cho et al. (1993), Science 261:1303; Carrell et al. (1994), Angew. Chem. Int. Ed. Engl. 33: 2059; Carell et al. (1994), Angew. Chem. Int. Ed. Engl. 33:2061; Gallop et al. (1994), J. Med. Chem. 37:1233; and Lam (1997), Anticancer Drug Des. 12:145.

Libraries of agents can be presented in solution (e.g., Houghten (1992), Bio/Techniques 13:412-421), or on beads (Lam (1991), Nature 354:82-84), chips (Fodor (1993), Nature 364:555-556), bacteria (U.S. Pat. No. 5,223,409), spores (U.S. Pat. No. 5,571,698; U.S. Pat. No. 5,403,484, and U.S. Pat. No. 5,223,409), plasmids (Cull et al. (1992), Proc. Natl. Acad. Sci. (USA) 89:1865-1869) or phage (Scott and Smith (1990), Science 249:386-390; Devlin (1990), Science 249:404-406; Cwirla et al. (1990), Proc. Natl. Acad. Sci. (USA) 87:6378-6382; and Felici (1991), J. Mol. Biol. 222:301-310).

### Compositions of the Present Invention

The invention also provides compositions, which may be used to modulate hemostasis.

### Antibodies

In one embodiment, the invention provides antibodies that antagonize CLEC-2 receptor activity. The term “antibody” refers to intact immunoglobulin molecules as well as to fragments thereof, such as Fab, F(ab′)₂, and Fv fragments, which are capable of binding an antigenic determinant. Antibodies that bind CLEC-2 polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies, which bind specifically to antigenic determinants (particular regions or three-dimensional structures on the protein). An antigenic determinant may compete with the intact antigen (i.e., the immunogen used to elicit the immune response) for binding to an antibody.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule.

The term “humanized antibody” refers to an antibody molecule in which the amino acid sequence in the non-antigen binding regions has been altered so that the antibody more closely resembles a human antibody, and still retains its original binding ability.

An “immunogenic fragment” is a polypeptide or oligopeptide fragment of CLEC-2, which is capable of eliciting an immune response when introduced into a living organism, for example, a mammal. The term “immunogenic fragment” also includes any polypeptide or oligopeptide fragment of CLEC-2, which is useful in any of the antibody production methods disclosed herein or known in the art.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies, which bind the ligand and prevent binding of the ligand to the receptor, as well as antibodies that bind the ligand, thereby preventing receptor activation, but do not prevent the ligand from binding the receptor. Further included in the invention are antibodies, which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1): 14-20 (1996) (which are all incorporated by reference herein in their entireties).

A preferred fragment produces an antibody that diminishes or completely prevents ligand binding. Antibodies can be developed against the entire receptor or portions of the receptor, for example, the intracellular amino terminal domain, the carboxy terminal extracellular domain, the entire transmembrane domain or specific transmembrane segments, any of the intracellular or extracellular loops, or any portions of these regions. Antibodies may also be developed against specific functional sites, such as the site of ligand binding, or sites that are glycosylated or phosphorylated.

Monoclonal antibodies to CLEC-2 may be prepared using any technique, which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell. Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce CLEC-2-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments, which contain specific binding sites for CLEC-2 may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between CLEC-2 and its specific antibody. A two-site, monoclonal-based immunoassay utilizing monoclonal antibodies reactive to two non-interfering CLEC-2 epitopes is generally used, but a competitive binding assay may also be employed (Pound, supra).

Various methods such as Scatchard analysis in conjunction with radioimmunoassay techniques may be used to assess the affinity of antibodies for CLEC-2. Affinity is expressed as an association constant, Kₐ, which is defined as the molar concentration of CLEC-2-antibody complex divided by the molar concentrations of free antigen and free antibody under equilibrium conditions. The Kₐ determined for a preparation of polyclonal antibodies, which are heterogeneous in their affinities for multiple CLEC-2 epitopes, represents the average affinity, or avidity, of the antibodies for CLEC-2. The Kₐ determined for a preparation of monoclonal antibodies, which are monospecific for a particular CLEC-2 epitope, represents a true measure of affinity. High-affinity antibody preparations with Kₐ ranging from about 10⁹ to 10¹² L/mole are preferred for use in immunoassays in which the CLEC-2-antibody complex must withstand rigorous manipulations. Low-affinity antibody preparations with Kₐ ranging from about 10⁶ to 10¹² L/mole are preferred for use in immunopurification and similar procedures which ultimately require dissociation of CLEC-2, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of CLEC-2-antibody complexes. Procedures for evaluating antibody specificity, titer, and avidity, and guidelines for antibody quality and usage in various applications, are generally available. (See, e.g., Catty, supra, and Coligan et al. supra.).

### A Pharmaceutical Composition Including CLEC-2

Also provided herein is a composition for reducing the severity of a pathological state mediated by a lack of CLEC-2 signaling. The composition includes a CLEC-2 substantially purified polypeptide or fragment therefore as defined herein or salt thereof and a pharmaceutical carrier as defined herein below. In another embodiment, the invention provides a nucleic acid encoding a CLEC-2 polypeptide or fragment thereof and a pharmaceutical carrier. The amino acid sequences of substantially purified CLEC-2 or nucleic encoding CLEC-2 are obtained from any species, particularly mammalian, including bovine, ovine, porcine, murine, equine, and preferably human, from any source whether natural, synthetic, semi-synthetic, or recombinant.

### Methods of Treatment for Hemostasis-Related Disorders

In another aspect, the present invention provides a method for reducing the severity of a pathological state mediated by CLEC-2 signaling. This method includes contacting a CLEC-2 receptor that includes a phosphorylated cytoplasmic domain, with a compound that blocks the binding of a signaling partner to the CLEC-2 receptor.

Preferably, the pathological state includes, but is not limited to, platelet activation.

Preferably, the signaling partner is a member of the Syk family of protein kinases.

In another embodiment, the present invention provides a method for reducing the severity of a pathological state mediated by CLEC-2 signaling comprising the method of administering to a mammal an antagonist of CLEC-2 signaling. Preferably, the pathological state includes, but is not limited to, acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

In another embodiment, the present invention provides a method for reducing the severity of a pathological state mediated by a lack of CLEC-2 signaling comprising the method of administering to a mammal an agonist of CLEC-2 signaling. Preferably, the pathological state is excessive bleeding.

The CLEC-2 receptor antagonists or agonists are administered as therapeutic compositions in physiologically and/or pharmaceutically acceptable carriers. For example, the CLEC-2 receptor antagonists or agonists can be administered to patients generally as described in Harrison's Principles of Internal Medicine, 14th Edition, McGraw-Hill, NY (1998). The characteristics of the carrier will depend on the route of administration.

Administration can be bolus, intermittent, or continuous, depending on the condition and response, as determined by those with skill in the art. In some preferred embodiments, the agonist is administered locally (e.g., intralesionally) and/or systemically. The term “local administration” refers to delivery to a defined area or region of the body, while the term “systemic administration” is meant to encompass delivery to the whole organism by oral ingestion, or by intramuscular, intravenous, subcutaneous, or intraperitoneal injection.

Such a composition can contain, in addition to the CLEC-2 receptor antagonist or agonist and carrier, diluents, fillers, salts, buffers, stabilizers, solubilizers, and other materials well known in the art. The pharmaceutical composition can also contain other active factors and/or agents, decrease cellular activation or increase cellular activation, preferably platelet activation. Such additional factors and/or agents can be included in the pharmaceutical composition to produce a synergistic effect with the CLEC-2 receptor antagonist or agonist, or to minimize side-effects caused by the antagonist or agonist.

The pharmaceutical compositions can be in the form of a liposome in which the CLEC-2 receptor antagonist or agonist is combined, in addition to other pharmaceutically acceptable carriers, with amphipathic agents such as lipids which exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. One particularly useful lipid carrier is lipofectin. Preparation of Such Liposomal Formulations is within the Level of Skill in the Art, as disclosed, for example, in U.S. Pat. No. 4,235,871; U.S. Pat. No. 4,501,728; U.S. Pat. No. 4,837,028; and U.S. Pat. No. 4,737,323. The pharmaceutical composition can further include compounds such as cyclodextrins and the like which enhance delivery of agents into cells, or slow release polymers.

In practicing the methods of treatment of the present invention, a therapeutically effective amount of one, two, or more CLEC-2 receptor antagonists or agonists is administered to a subject afflicted with a disease or disorder characterized by increased or decreased platelet aggregation. The antagonists or agonists can be administered in accordance with the method of the invention either alone or in combination with other known therapies for the disease or disorder. When co-administered with one or more other therapies, the CLEC-2 receptor antagonist or agonist can be administered either simultaneously with the other treatment(s), or sequentially. If administered sequentially, the attending physician will decide on the appropriate sequence of administering the CLEC-2 receptor antagonist or agonist in combination with the other therapy.

Administration of the CLEC-2 receptor antagonist or agonist in a pharmaceutical composition to practice the methods of the invention can be carried out in a variety of conventional ways, such as implantation, oral ingestion, inhalation, or cutaneous, subcutaneous, intramuscular, or intravenous injection.

If CLEC-2 receptor antagonists or agonists are administered locally or regionally (e.g., to the site of a blood clot) as opposed to systemically, normal tissue uptake should be reduced. CLEC-2 receptor antagonists or agonists may be administered locally or regionally by admixing the antagonist or agonist in a biocompatible matrix material and implanting or applying the mixture at or near a site to be treated. For example, biocompatible polymers such as polylactic acid (PLA), polyglycolic acid (PGA), poly(co-lactic-glycolic) acid (PLGA), poly(ethylene glycol) (PEG), and the like can be formed into bioerodable implants which contain a CLEC-2 receptor antagonist or agonist. Alternatively, various poultices or waxes known in the art can be admixed with a CLEC-2 receptor antagonist or agonist and applied to a site within the body (e.g., the site of a blood clot). In addition, methods of encapsulating CLEC-2 receptor antagonists or agonists in liposomes and targeting these liposomes to selected tissues by inserting proteins into the liposome surface can be utilized (Pagnan et al. (2000), J. Natl. Can. Inst. 92:253-61; Yu et al. (1999), Pharm. Res. 16:1309-15).

When a therapeutically effective amount of a CLEC-2 receptor antagonist or agonist is administered orally, the CLEC-2 receptor antagonist or agonist will be in the form of a tablet, capsule, powder, solution or elixir. When administered in tablet form, the pharmaceutical composition can additionally contain a solid carrier such as a gelatin or an adjuvant. The tablet, capsule, and powder can contain from about 5 to 95% CLEC-2 receptor antagonist or agonist and preferably from about 25 to 90% CLEC-2 receptor antagonist or agonist. When administered in liquid form, a liquid carrier such as water, petroleum, oils of animal or plant origin such as peanut oil, mineral oil, soybean oil, sesame oil, or synthetic oils can be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the CLEC-2 receptor antagonist or agonist and preferably from about 1 to 50% CLEC-2 receptor agonists.

When a therapeutically effective amount of a CLEC-2 receptor antagonist or agonist is administered by intravenous, subcutaneous, intramuscular, or intraperitoneal injection, the CLEC-2 receptor antagonist or agonist will be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, subcutaneous, intramuscular, or intraperitoneal injection should contain, in addition to the CLEC-2 receptor antagonist or agonist, an isotonic vehicle. The pharmaceutical composition can also contain stabilizers, preservatives, buffers, antioxidants, or other additives known to those of skill in the art.

The amount of CLEC-2 receptor antagonist or agonist in the pharmaceutical composition will depend upon the nature and severity of the condition being treated, and on the nature of prior treatments, which the patent has undergone. Ultimately, the attending physician will decide the amount of CLEC-2 receptor antagonist or agonist with which to treat each individual patient. Similarly, the duration of intravenous therapy using the pharmaceutical composition will vary depending on the severity of the disease being treated and the condition and potential idiosyncratic response of each individual patient. Ultimately the attending physician will decide on the appropriate duration of intravenous therapy using the pharmaceutical composition of the present invention. Initially, the attending physician can administer low doses of the CLEC-2 receptor antagonist or agonist and observe the patient's response. Larger doses of CLEC-2 receptor antagonist or agonists can be administered until the optimal therapeutic effect is obtained for the patient, and at that point the dosage is not increased further.

It is contemplated that the various pharmaceutical compositions used to practice the methods of the present invention should contain about 10 μg to about 20 mg of CLEC-2 receptor antagonist or agonists per kg body or organ weight.

### EXAMPLES

In order that the invention described herein may be more fully understood, the following examples are set forth. It should be understood that these examples are for illustrative purposes only and are not to be construed as limiting the present invention in any manner.

### Example 1

### Materials and Methods Used to Determine Signal Transduction of CLEC-2

### a) Animals and Materials

Genetically modified mice deficient in PLCγ2¹⁷, Syk¹⁸, LAT¹⁹, SLP-76²⁰, and Vav1/3²¹ were obtained as previously described. Rhodocytin was purified from the venom of Calloselasma rhodostoma^{11,22}. Polyclonal anti-CLEC-2 antibody was from R&D Systems Inc. (Minneapolis, Minn., USA). Polyclonal rabbit anti-rhodocytin antibody was generated by Takashi Morita and Gavin Laing. Gly-Arg-Gly-Asp-Ser (GRGDS) peptide (SEQ ID NO: 5) was obtained from Peptide Institute (Osaka, Japan). Ser-Phe-Leu-Leu-Arg-Asn (SFLLRN) peptide (SEQ ID NO: 6) (thrombin receptor agonist peptide, TRAP) was from Bachem Biochemica (Heidelberg, Germany). Biotin-labelled phosphorylated or non-phosphorylated peptide containing the YXXL motif in CLEC-2 (Biotin-6-aminohexanoic acid (Ahx)-MQDEDGY(PO₃H₂)ITLNIK-OH, (SEQ ID NO: 7) or Biotin-6-aminohexanoic acid (Ahx)-MQDEDGYITLNIK-OH) (SEQ ID NO: 7) was synthesised by Kurabo Industries Ltd. (Osaka, Japan). All other reagents were from previously named sources^{6,17,21}.

### b) Platelet Preparation

Venous blood from healthy drug-free volunteers was taken into 10% sodium citrate. Murine blood was drawn from CO₂ terminally-narcosed mice by portal vein puncture and taken into 100 μl of acid citrate dextrose. Washed human and murine platelets were obtained by centrifugation as previously described, using prostacyclin to prevent activation during the isolation procedure¹⁷. Both sets of washed platelets were resuspended in modified Tyrode's buffer¹⁷ at a cell density of 2×10⁸ or 1×10⁹/ml.

c) Pull-Down with Rhodocytin-Coated Beads

200 μg of purified rhodocytin was covalently coupled to 122 mg of CNBr-activated Sepharose 4B beads according to the manufacturer's instructions. Glycine-coated beads were used as a negative control. Surface proteins in washed platelets or rhodocytin were labeled with biotin using ECL Protein Biotinylation System (Amersham Biosciences Corp., Piscataway, N.J.). 1 ml of biotin labeled- or unlabeled-washed platelets (1×10⁹/ml) was lysed by an equal volume of 2× ice-cold lysis buffer 17 and precleared by 100 μl of Sepharose 4B (50% slurry) for 1 hour. After the detergent-insoluble debris was clarified by centrifugation at 16000 g for 10 min, the supernatant was incubated with 200 μl of rhodocytin-bound or glycine-bound Sepharose 4B for 4 hours at 4° C. The beads were washed 5 times in 1× lysis buffer, and proteins were eluted from the beads with 40 μl of SDS reducing sample buffer and boiled for 5 min. Precipitated platelet proteins were separated by 4-20% SDS-PAGE and electrotransferred to PVDF membrane. Biotin-labeled surface proteins were detected by HRP (horse radish peroxidase)-conjugated streptavidin. Unlabeled platelet proteins electrotransferred to PVDF membrane were ligand-blotted with biotin-labeled rhodocytin and HRP-conjugated streptavidin, followed by reprobing with anti-CLEC-2 antibody. For mass spectrometric analysis, unlabelled washed platelets were used and gels were stained with Coomassie Brilliant Blue.

### d) Protein Digestion and Mass Spectrometric Analysis.

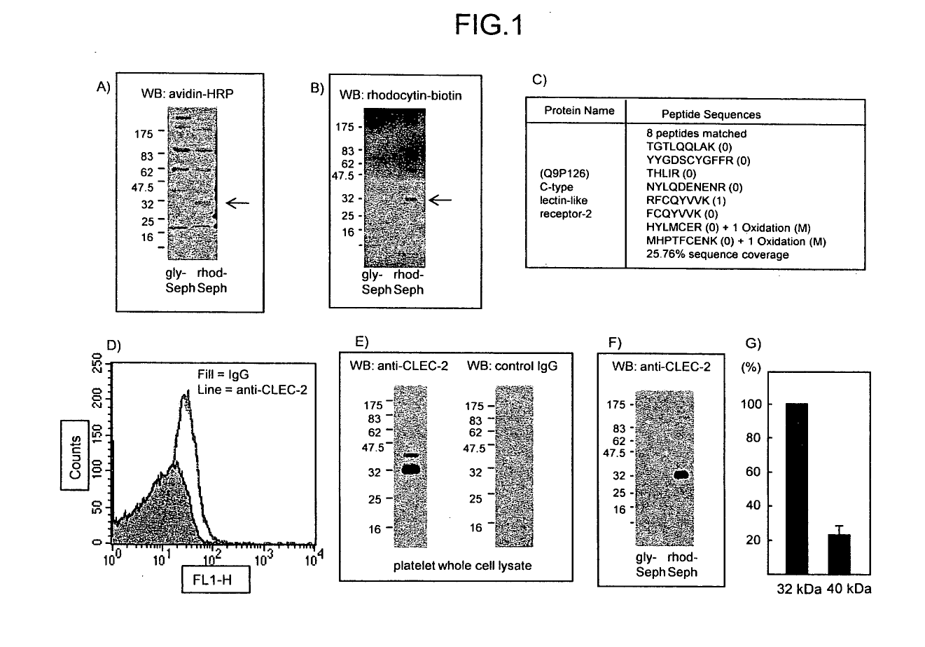

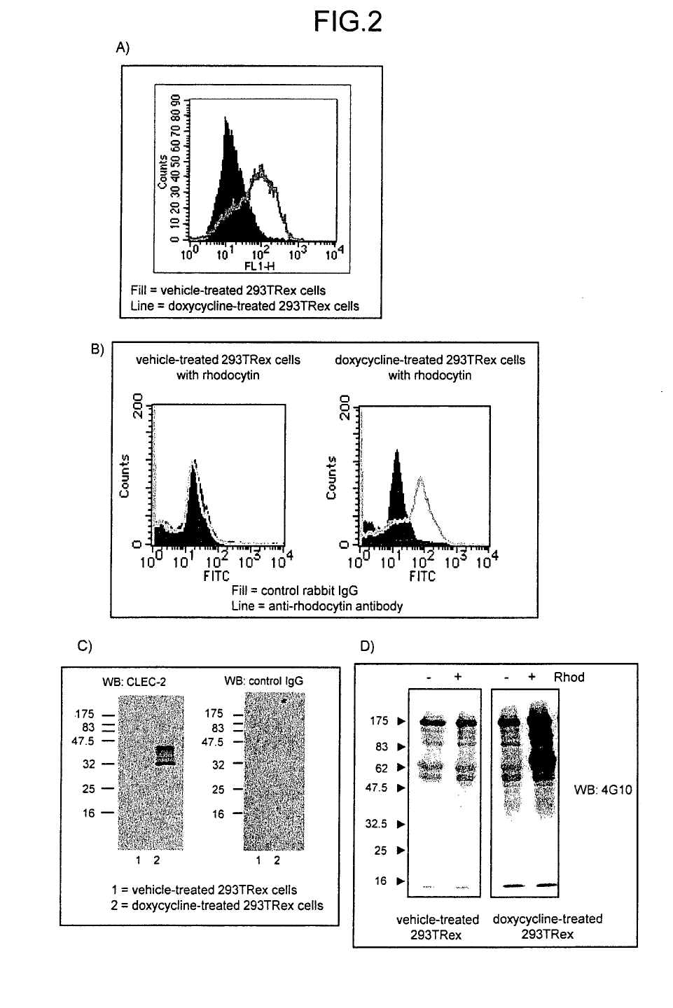

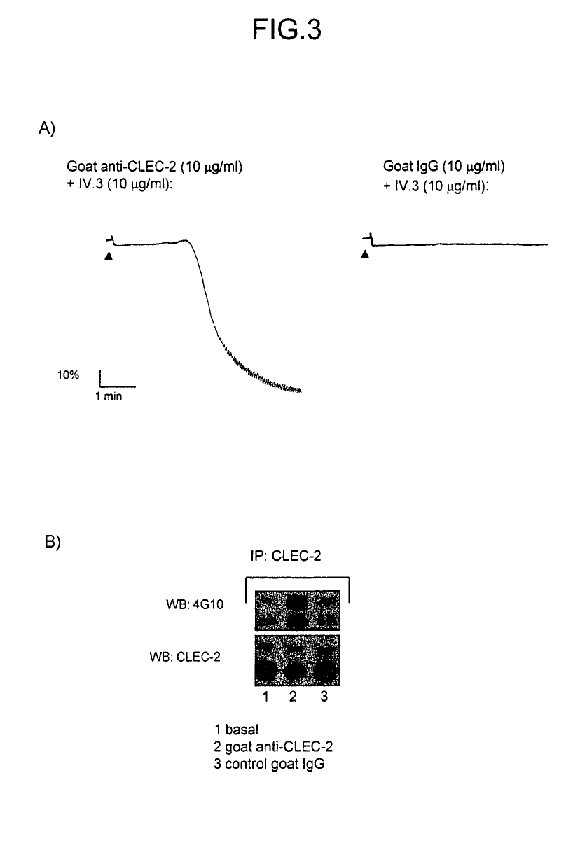

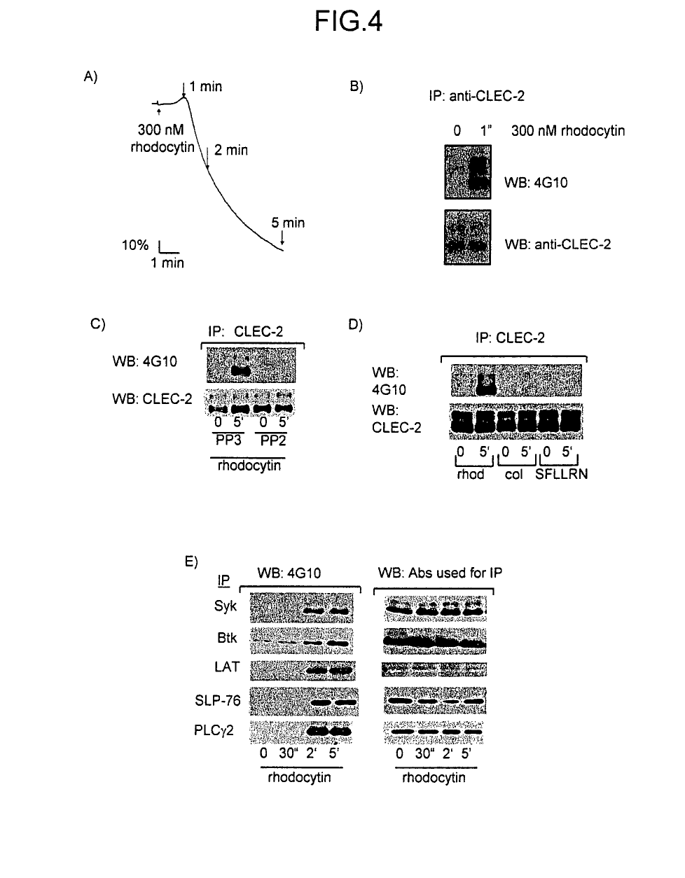

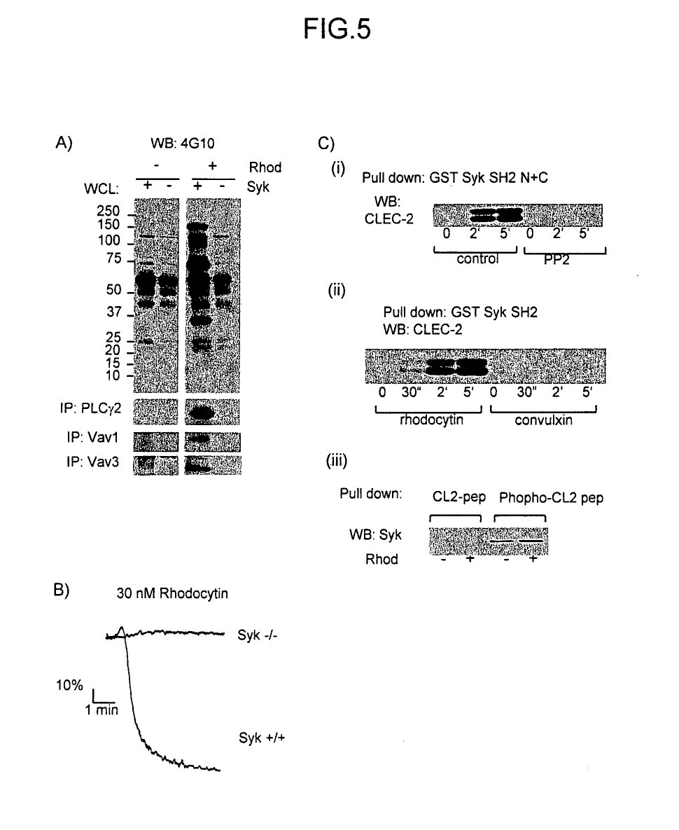

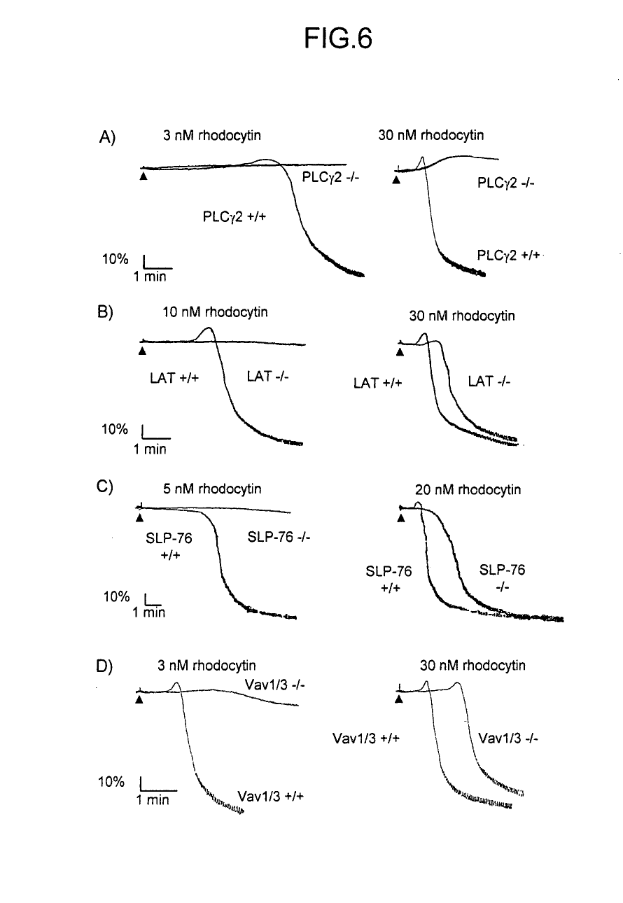

The gel pieces were dried in a speed-vac and in-gel trypsinolysis was carried out as described²³. The HPLC (CapLC, Waters, Milford, Mass.) was coupled to a Q-TOF mass spectrometer (Micromass, Manchester, UK) equipped with a nanoelectrospray Z-spray source. Mobile phases for the HPLC were 5% acetonitrile, 0.1% formic acid (Solvent A) and 95% acetonitrile, 0.1% formic acid (Solvent B). The tryptic peptides were loaded and desalted on a 300 μm id/5 mm length C18 PepMap pre-column (LC packings, San Francisco, Calif.), and then eluted at a flow of 200 nl/min onto a 75 μm id/25 cm length C18 PepMap nano column, using a gradient of 5-40% Solvent B in 40 min and then 40-80% Solvent B in 3 min. MS/MS analysis was carried out as previously described²⁴. The data base search was performed with the MASCOT search tool (Matrix Science, London, UK) screening SWISS-PROT. FIG. 1 (c) depicts the compiled data output file for Mascot MS Searches Swiss-Prot Search results. The eight matched peptides include TGTLQQLAK (SEQ ID NO: 8), YYGDSCYGFFR (SEQ ID NO: 9), THLIR (SEQ ID NO: 10), NYLQDENENR (SEQ ID NO: 11), RFCQVVVK (SEQ ID NO: 12), FCQYVVK (SEQ ID NO: 13), HYLMCER (SEQ ID NO: 14), AND MHPTFCENK (SEQ ID NO: 15).

### e) Cell Culture and Stimulation

CLEC-2 was expressed under a tet repressor protein in 293T-REx™ cell line (Invitrogen Corp., Carlsbad, Calif.) and were grown as described²⁵. CLEC-2 expression was induced by addition of 1 μg/ml of doxycycline to the medium 24-48 hours before experimentation. Vehicle-added cells were used for control. Cells were detached and resuspended in modified Tyrode's buffer as described⁶ and stimulated with vehicle or 500 nM rhodocytin for 10 min at 37° C. Reactions were stopped by addition of 2×SDS sample buffer. Tyrosine phosphorylation and CLEC-2 expression were detected by anti-phosphotyrosine (4G10) and anti-CLEC-2 antibodies, respectively as described¹⁷.

### f) Flow Cytometry Studies

Washed human platelets (1×10⁸/ml) or 293T-REx™ cells (5×10⁶/ml) were incubated with 2 μg/ml of anti-CLEC-2 antibody for 15 min followed by staining with FITC-conjugated anti-goat IgG for 15 min at room temperature. After diluting three folds with PBS, samples were analyzed with a FACScan flow cytometer and CellQuest software (Becton Dickinson, San Jose, Calif.) as described¹⁷. To detect direct binding of rhodocytin, 293T-REx™ cells (5×10⁶/ml) pretreated with or without doxycycline were preincubated with 100 nM rhodocytin for 15 min at room temperature. After excess of rhodocytin were removed by centrifugation, cells were incubated with 10 μg/ml of control rabbit IgG or anti-rhodocytin antibody for 20 min at room temperature followed by removing unbound antibodies. Then, the samples were stained with FITC-conjugated anti-rabbit IgG and analyzed as described above.

### g) Immunoprecipitation and Western Blotting

Washed human platelets (1×10⁹/ml) pretreated with 1 mM GRGDS (SEQ ID NO: 5) peptide or washed murine platelets (2×10⁸/ml) pretreated with 10 μM lotrafiban to inhibit platelet aggregation were stimulated with 50 nM rhodocytin, 100 μM SFLLRN (SEQ ID NO: 6), or 50 μg/ml of collagen. Where indicated, 30 μM PP2 (a Src kinase inhibitor) or PP3 (an inactive analogue of PP2) were incubated for 10 min at 37° C. Reactions were terminated by addition of 2× ice-cold lysis buffer. Platelet lysates were precleared and detergent-insoluble debris was clarified as described 17. A small aliquot was dissolved with SDS sample buffer for detection of total tyrosine phosphorylation. Antibodies against CLEC-2, PLCγ2, Syk, Btk, SLP-76, LAT, Vav1, or Vav3 were added to the resultant supernatant and incubated overnight. Where indicated, 10 μg of CLEC-2 YXXL-containing peptide plus avidin-Sepharose or GST-fusion protein-associated glutathione Sepharose were incubated with the lysates. In the indicated experiments, human washed platelets (1×10⁹/ml) were stimulated by 10 μg/ml of anti-CLEC-2 antibody or control goat IgG in the presence of F(ab)′₂ fragment of anti-FcγRIIA antibody (IV.3, 10 μg/ml). The samples were lysed with 2× lysis buffer before and 5 min after stimulation. Five μg/ml of anti-CLEC-2 antibody and control goat IgG were added to the sample lysed before stimulation. Five μg/ml of anti-CLEC-2 or control goat IgG was added to the sample stimulated by control IgG and that stimulated by CLEC-2 antibody, respectively. After removing insoluble debris by centrifugation, CLEC-2 was immunoprecipitated by addition of protein G. Precipitated proteins or whole cell lysates were separated by SDS-PAGE, electrotransferred, and western blotted by the indicated antibodies.

### h) Platelet Aggregation

Human washed platelets (2×10⁸/ml) were stimulated by 10 μg/ml of anti-CLEC-2 antibody or control goat IgG in the presence of F(ab)′₂ fragment of anti-FcγRIIA antibody (IV.3, 10 μg/ml). Washed murine or human (2×10⁸/ml) platelets stimulated with low and high concentrations of rhodocytin as indicated in the text. Aggregation was monitored by light transmission using a Born aggregometer as described¹⁷.

### Example 2

### CLEC-2 Binds to Rhodocytin-Coated Beads

We used Sepharose 4B beads that were covalently coupled with rhodocytin or glycine to isolate binding proteins for the snake toxin from platelet lysates, whose surface proteins had been labelled with biotin. Blotting using streptavidin demonstrated that the rhodocytin- but not glycine-coupled Sepharose 4B beads precipitated a 32 kDa protein, that was pulled down along with several other proteins that associated non-specifically with both sets of beads (FIG. 1A). The same 32 kDa band was also precipitated by rhodocytin- but not glycine-coupled beads from non-biotin labelled platelets, as detected by ligand blotting using rhodocytin (FIG. 1B). The portion of the gel corresponding to the 32 kDa band was excised, digested with trypsin and subjected to MS/MS analysis. This approach identified CLEC-2 as a component of the 32 kDa band (FIG. 1C). The expression of CLEC-2 on platelets was confirmed by flow cytometry and western blotting using a specific antibody (FIGS. 1D and E). Interestingly, CLEC-2 was detected as a doublet in platelets by western blotting, with a major band migrating at 32 kDa and a minor band at 40 kDa (FIG. 1E). The former corresponds to that precipitated by the rhodocytin-coupled beads. The apparent absence of the 40 kDa band in the rhodocytin-affinity eluates may be because of comigration with a non-specific protein (FIG. 1A only) or because of a lower level of expression of the 40 kDa band combined with a limitation in the sensitivity of the ligand blotting assay. Significantly, precipitation of 32 and 40 kDa bands by rhodocytin- but not by glycine-coupled beads could be detected using a specific antibody to CLEC-2, although the 40 kDa band was present at a much lower level than the 32 kDa band, consistent with the results in platelets (FIG. 1F). Densitometric analysis of anti-CLEC-2 western blots from several platelet samples demonstrated that the level of expression of the 40 kDa is 23.1±5.3% of 32 kD band (FIG. 1G).

These results identify CLEC-2 as a novel rhodocytin binding protein on the platelet surface. Intriguingly, CLEC-2 is a type II transmembrane protein of the C-type lectin superfamily with a single tyrosine residue in its cytoplasmic tail that is present in a YXXL motif²⁶ suggesting that it may have the capacity to signal through a tyrosine kinase-based pathway. There are no previous reports of the presence of CLEC-2 on platelets or its ability to function as a signaling receptor.

### Example 3

### Rhodocytin Stimulates Tyrosine Phosphorylation in a CLEC-2 Expressing Cell Line

To confirm CLEC-2 as a receptor for rhodocytin and its ability to generate tyrosine kinase-based intracellular signals, we used a previously reported cell line in which CLEC-2 was expressed under the control of a tet repressor protein²⁵. The addition of doxycycline to transfected 293T-REx™ cells induces surface expression of CLEC-2, as measured using a specific antibody to the lectin receptor (FIG. 2A) or a combination of rhodocytin and a rhodocytin antibody (FIG. 2B). Further, western blotting for CLEC-2 using a specific antibody but not with a control IgG identified two major bands of approximately 32 and 40 kDa and a minor band of 34 kDa in doxycycline-treated but not control cells (FIG. 2C). The presence of more than one band corresponding to CLEC-2 is similar to the situation in platelets, although three bands were detected in the cell line and the 40 kDa band was expressed at a similar level to the 32 kDa band. Significantly, rhodocytin stimulation induced an increase in tyrosine phosphorylation of several proteins in whole cell lysates from doxycycline-treated (CLEC-2-expressing) but not from vehicle-treated cells (FIG. 2D). Together, these results confirm that CLEC-2 is a functional receptor for rhodocytin.

### Example 4

### Activation of Platelets Using an Antibody to CLEC-2

We used an antibody to CLEC-2 to investigate whether the lectin receptor is able to induce activation of platelets in the absence of signals from other receptors. This experiment is necessary bearing in mind that rhodocytin binds to at least two other platelet receptors, the integrin α2β1 and GPIbα^{12,13,14}. As shown in FIG. 3A, an antibody to CLEC-2 induces platelet aggregation after a characteristic delay. This experiment was carried out in the presence of monoclonal antibody IV.3 to block binding of Fc portion of the antibody to FcγRIIA. A control goat IgG did not stimulate aggregation. Significantly, the CLEC-2 antibody, but not a control goat IgG, induced a significant increase in tyrosine phosphorylation above basal of both the 32 and 40 kDa forms of CLEC-2, as demonstrated by immunoprecipitation of CLEC-2 and western blotting for phosphotyrosine and then for CLEC-2 (FIG. 3B). Since CLEC-2 contains only a single tyrosine in its cytosolic tail, phosphorylation must have taken place at this YXXL motif. These results demonstrate that engagement of CLEC-2 is sufficient to mediate platelet activation in the absence of signals from other receptors. Further, the demonstration that the lectin receptor undergoes tyrosine phosphorylation provides a possible mechanism underlying CLEC-2-mediated platelet activation.

### Example 5

### Src Kinases Mediate Tyrosine Phosphorylation of CLEC-2

Rhodocytin stimulates platelet activation with a characteristic lag phase, which reduces as the concentration of the toxin is increased. At a relatively high concentration of rhodocytin (300 nM), activation begins after a delay of approximately 30 sec (FIG. 4A), although this is much longer with lower concentrations of the snake venom toxin. In line with this, the stimulation of tyrosine phosphorylation in whole cell lysates by rhodocytin occurs over a similar time scale to that of platelet aggregation, occurring more rapidly with higher concentrations of rhodocytin (not shown). Significantly, substantial phosphorylation of CLEC-2 can be seen in response to rhodocytin at 60 sec, which corresponds to the peak in the shape change response and the onset of aggregation (FIG. 4A, B). This is reminiscent of the time course of platelet aggregation by collagen, which also occurs after a dose-dependent delay, and together with the increase in tyrosine phosphorylation of platelets lysates and Fc receptor γ-chain, which forms part of the collagen receptor²⁷.

The significance of tyrosine phosphorylation events in platelet activation by rhodocytin is illustrated by the ability of the Src family kinase inhibitors PP1 or PP2 to block tyrosine phosphorylation in whole cells and all functional responses in response to stimulation by rhodocytin¹³ (not shown). Significantly, PP2 but not its inactive analogue PP3, also block tyrosine phosphorylation of both the 32 and 40 kDa forms of CLEC-2 (FIG. 4C), consistent with the possibility that tyrosine phosphorylation of CLEC-2 initiates platelet activation. In contrast, CLEC-2 does not undergo tyrosine phosphorylation in response to stimulation by collagen or by a thrombin receptor PAR1-activating peptide, SFLLRN (SEQ ID NO: 6) over a time course of up to 5 min (FIG. 4D), demonstrating that the lectin receptor is only phosphorylated upon direct activation.

We have previously reported that rhodocytin stimulates tyrosine phosphorylation of Syk in platelets¹³. The observation that CLEC-2 undergoes tyrosine phosphorylation on a single YXXL motif, which forms one half of an ITAM sequence, raises the possibility that the lectin receptor signals through recruitment of Syk to the cytosolic tail and initiating a signaling cascade that is similar to that used by the collagen receptor GPVI. In line with this, rhodocytin stimulates tyrosine phosphorylation of Syk and many of the major signaling proteins in the GPVI cascade, including the tyrosine kinase Btk, the adapters LAT and SLP-76, the GTP exchange factors Vav1 and Vav3, and PLCγ2 (FIGS. 4E and 5A). The time course of phosphorylation of all of these proteins is similar to that for Syk (FIG. 4E). Tyrosine phosphorylation of all of these proteins is abolished in the presence of the Src kinase inhibitor PP2 (not shown).

### Example 6

### Syk Mediates Platelet Activation by Rhodocytin

The functional role of Syk in signaling by rhodocytin was further investigated using murine platelets deficient in the tyrosine kinase. Rhodocytin stimulates robust tyrosine phosphorylation in whole cell lysates of murine platelets, which is completely inhibited in the absence of Syk (FIG. 5A). The critical role of Syk in signaling by rhodocytin is further illustrated by the complete loss of phosphorylation of PLCγ2 and the two members of the Vav family, Vav1 and Vav3, in the Syk-deficient platelets (FIG. 5A). Furthermore, shape change and aggregation induced by rhodocytin are completely inhibited in the absence of Syk (FIG. 5B). These results thereby confirm a proximal role for Syk in platelet activation by rhodocytin.

The possibility that tyrosine phosphorylation of CLEC-2 leads to recruitment of Syk, thereby initiating the downstream tyrosine phosphorylation signaling cascade, was investigated using a GST fusion protein encoding the tandem SH2 domains of Syk²⁸. The Syk fusion protein precipitated similar levels of the 32 and 40 kDa forms of CLEC-2 from stimulated but not control platelets, whereas the ability to precipitate CLEC-2 was abolished in the presence of the Src kinase inhibitor, PP2 (FIG. 5Ci). Further, the Syk fusion protein was able to precipitate CLEC-2 within 30 sec of stimulation by rhodocytin, showing that it occurs in parallel with the onset of shape change/aggregation, but not by the GPVI agonist convulxin (FIG. 5C ii), confirming the specificity of regulation. In line with these results, Syk could be precipitated by a phosphorylated peptide containing the YXXL motif from the cytosolic tail of CLEC-2, whereas there was no interaction with the equivalent, non-phosphorylated peptide (FIG. 5Ciii). These results thereby support a model in which Syk interacts with the phosphorylated tail of CLEC-2 thereby initiating downstream signaling events. We have not been unable to confirm this association in stimulated platelets, however, through immunoprecipitation studies using antibodies to CLEC-2 or to Syk, possibly because of a relatively low affinity of the Syk SH2 domain for a single phospho-YXXL motif, thereby rendering the complex unstable on solubilisation.

### Example 7

### Critical Role of LAT, SLP-76, Vav1/3, and PLCγ2 in Signaling by Rhodocytin

We used platelets from mutant mice to establish the role of a number of the proteins that undergo tyrosine phosphorylation upon stimulation by rhodocytin. Low (3-10 nM) and intermediate (20-30 nM) concentrations of rhodocytin were used to establish whether any inhibitory effects could be overcome with increasing concentrations of the snake toxin. The response to a low concentration of rhodocytin was abolished in the absence of PLCγ2, whereas an intermediate concentration induces shape change, which is likely to reflect the presence of a low level of PLCγ1¹⁷ (FIG. 6A). A marked inhibition of the response to a low concentration of rhodocytin was also seen in the absence of LAT, SLP-76, and Vav1/Vav3, although an almost full recovery of aggregation was seen in response to higher concentrations of the snake toxin (FIG. 6B-D). The results with LAT and PLCγ2 are similar to those obtained following stimulation of mutant murine platelets by GPVI, using either the snake venom toxin convuixin or the GPVI-specific collagen related peptide (CRP) as agonists^{17,19}. In contrast, the observation that a high concentration of rhodocytin is able to stimulate maximal aggregation in the absence of SLP-76, or the combined absence of Vav1 and Vav3^{29,21}, contrasts with those seen for convulxin or CRP, where aggregation is largely abolished. These observations therefore further distinguishing the CLEC-2-dependent signaling pathway from that used by the collagen receptor, GPVI.

The following citations are referred to throughout the examples to better describe the present invention and are herein incorporated by reference.

### REFERENCES

1. Andrews R K, Kamiguti A S, Berlanga O, Leduc M, Theakston R D, Watson S P. The use of snake venom toxins as tools to study platelet receptors for collagen and von Willebrand factor. Haemostasis. 2001; 31:155-172
2. Morita T. Structures and functions of snake venom CLPs (C-type lectin-like proteins) with anti-coagulant-, procoagulant-, and platelet-modulating activities. Toxicon. In press
3. Clemetson J M, Polgar J, Magnenat E, Wells T N, Clemetson K J. The platelet collagen receptor glycoprotein VI is a member of the immunoglobulin superfamily closely related to FcalphaR and the natural killer receptors. J Biol Chem. 1999; 274:29019-29024
4. Polgar J, Clemetson J M, Kehrel B E, Wiedemann M, Magnenat E M, Wells T N, Clemetson K J. Platelet activation and signal transduction by convulxin, a C-type lectin from Crotalus durissus terrificus (tropical rattlesnake) venom via the p62/GPVI collagen receptor. J Biol Chem. 1997; 272:13576-13583
5. Jandrot-Perrus M, Lagrue A H, Okuma M, Bon C. Adhesion and activation of human platelets induced by convulxin involve glycoprotein VI and integrin alpha2beta1. J Biol Chem. 1997; 272:27035-27041
6. Suzuki-Inoue K, Tulasne D, Shen Y, Bori-Sanz T, Inoue O, Jung S M, Moroi M, Andrews R K, Berndt M C, Watson S P. Association of Fyn and Lyn with the proline-rich domain of glycoprotein VI regulates intracellular signaling. J Biol Chem. 2002; 277:21561-21566
7. Bori-Sanz T, Inoue K S, Berndt M C, Watson S P, Tulasne D. Delineation of the region in the glycoprotein VI tail required for association with the Fc receptor gamma-chain. J Biol Chem. 2003; 278:35914-35922
8. Lee W H, Du X Y, Lu Q M, Clemetson K J, Zhang Y. Stejnulxin, a novel snake C-type lectin-like protein from Trimeresurus stejnegeri venom is a potent platelet agonist acting specifically via GPVI. Thromb Haemost. 2003; 90:662-671
9. Asazuma N, Marshall S J, Berlanga O, Snell D, Poole A W, Berndt M C, Andrews R K, Watson S P. The snake venom toxin alboaggregin-A activates glycoprotein VI. Blood. 2001; 97:3989-3991
10. Andrews R K, Gardiner E E, Asazuma N, Berlanga O, Tulasne D, Nieswandt B, Smith A I, Berndt M C, Watson S P. A novel viper venom metalloproteinase, alborhagin, is an agonist at the platelet collagen receptor GPVI. J Biol Chem. 2001; 276:28092-28097
11. Shin Y, Morita T. Rhodocytin, a functional novel platelet agonist belonging to the heterodimeric C-type lectin family, induces platelet aggregation independently of glycoprotein Ib. Biochem Biophys Res Commun. 1998; 245:741-745
12. Huang T F, Liu C Z, Yang S H. Aggretin, a novel platelet-aggregation inducer from snake (Calloselasma rhodostoma) venom, activates phospholipase C by acting as a glycoprotein Ia/IIa agonist. Biochem J. 1995; 309 (Pt 3): 1021-1027
13. Suzuki-Inoue K, Ozaki Y, Kainoh M, Shin Y, Wu Y, Yatomi Y, Ohmori T, Tanaka T, Satoh K, Morita T. Rhodocytin induces platelet aggregation by interacting with glycoprotein Ia/IIa (GPIa/IIa, Integrin alpha 2beta 1). Involvement of GPIa/IIa-associated src and protein tyrosine phosphorylation. J Biol Chem. 2001; 276:1643-1652
14. Navdaev A, Clemetson J M, Polgar J, Kehrel B E, Glauner M, Magnenat E, Wells T N, Clemetson K J. Aggretin, a heterodimeric C-type lectin from Calloselasma rhodostoma (malayan pit viper), stimulates platelets by binding to alpha 2beta 1 integrin and glycoprotein Ib, activating Syk and phospholipase Cgamma 2, but does not involve the glycoprotein VI/Fc receptor gamma chain collagen receptor. J Biol Chem. 2001; 276:20882-20889
15. Bergmeier W, Bouvard D, Eble J A, Mokhtari-Nejad R, Schulte V, Zirngibl H, Brakebusch C, Fassler R, Nieswandt B. Rhodocytin (aggretin) activates platelets lacking alpha(2)beta(1) integrin, glycoprotein VI, and the ligand-binding domain of glycoprotein Ibalpha. J Biol Chem. 2001; 276:25121-25126
16. Suzuki-Inoue K, Garcia A, Eble J A, Fuller G, Pohlmann S, Zitzmann N, Inoue O, Gartner T K, Morita T, Ozaki Y, Watson S P. Identification of CLEC-2 as a Novel Signalling Receptor for Rhodocytin in Platelets [abstract]. J Thromb Haemost. 2005 Abstract P1494 in press
17. Suzuki-Inoue K, Inoue O, Frampton J, Watson S P. Murine GPVI stimulates weak integrin activation in PLC{gamma}2−/− platelets: involvement of PLC{gamma} 1 and PI 3-kinase. Blood. 2003; 102:1367-1373
18. Poole A, Gibbins J M, Turner M, van Vugt M J, van de Winkel J G, Saito T, Tybulewicz V L, Watson S P. The Fc receptor gamma-chain and the tyrosine kinase Syk are essential for activation of mouse platelets by collagen. EMBO J. 1997; 16:2333-2341
19. Pasquet J M, Gross B, Quek L, Asazuma N, Zhang W, Sommers C L, Schweighoffer E, Tybulewicz V, Judd B, Lee J R, Koretzky G, Love P E, Samelson L E, Watson S P. LAT is required for tyrosine phosphorylation of phospholipase cgamma2 and platelet activation by the collagen receptor GPVI. Mol Cell Biol. 1999; 19:8326-8334
20. Clements J L, Lee J R, Gross B, Yang B, Olson J D, Sandra A, Watson S P, Lentz S R, Koretzky G A. Fetal hemorrhage and platelet dysfunction in SLP-76-deficient mice. J Clin Invest. 1999; 103:19-25
21. Pearce A C, Senis Y A, Billadeau D D, Turner M, Watson S P, Vigorito E. Vav1 and vav3 have critical but redundant roles in mediating platelet activation by collagen. J Biol Chem. 2004; 279:53955-53962
22. Eble J A, Beermann B, Hinz H J, Schmidt-Hederich A. alpha 2beta 1 integrin is not recognized by rhodocytin but is the specific, high affinity target of rhodocetin, an RGD-independent disintegrin and potent inhibitor of cell adhesion to collagen. J Biol Chem. 2001; 276:12274-12284
23. Shevchenko A, Jensen O N, Podtelejnikov A V, Sagliocco F, Wilm M, Vorm O, Mortensen P, Shevchenko A, Boucherie H, Mann M. Linking genome and proteome by mass spectrometry: large-scale identification of yeast proteins from two dimensional gels. Proc Natl Acad Sci USA. 1996; 93:14440-14445
24. Garcia A, Prabhakar S, Hughan S, Anderson T W, Brock C J, Pearce A C, Dwek R A, Watson S P, Hebestreit H F, Zitzmann N. Differential proteome analysis of TRAP-activated platelets: involvement of DOK-2 and phosphorylation of RGS proteins. Blood. 2004; 103:2088-2095
25. Pohlmann S, Zhang J, Baribaud F, Chen Z, Leslie G J, Lin G, Granelli-Piperno A, Doms R W, Rice C M, McKeating J A. Hepatitis C virus glycoproteins interact with DC-SIGN and DC-SIGNR. J Virol. 2003; 77:4070-4080
26. Colonna M, Samaridis J, Angman L. Molecular characterization of two novel C-type lectin-like receptors, one of which is selectively expressed in human dendritic cells. Eur J Immunol. 2000; 30:697-704
27. Asselin J, Gibbins J M, Achison M, Lee Y H, Morton L F, Farndale R W, Barnes M J, Watson S P. A collagen-like peptide stimulates tyrosine phosphorylation of syk and phospholipase C gamma2 in platelets independent of the integrin alpha2beta1. Blood. 1997; 89:1235-1242
28. Gibbins J, Asselin J, Farndale R, Barnes M, Law C L, Watson S P. Tyrosine phosphorylation of the Fc receptor gamma-chain in collagen-stimulated platelets. J Biol Chem. 1996; 271:18095-18099
29. Judd B A, Myung P S, Obergfell A, Myers E E, Cheng A M, Watson S P, Pear W S, Allman D, Shattil S J, Koretzky G A. Differential requirement for LAT and SLP-76 in GPVI versus T cell receptor signaling. J Exp Med. 2002; 195:705-717
30. Chung C H, Wu W B, Huang T F. Aggretin, a snake venom-derived endothelial integrin alpha 2 beta 1 agonist, induces angiogenesis via expression of vascular endothelial growth factor. Blood. 2004; 103:2105-2113
31. Marshall A S, Gordon S. Commentary: C-type lectins on the macrophage cell surface—recent findings. Eur J Immunol. 2004; 34:18-24
32. Cambi A, Figdor C G. Dual function of C-type lectin-like receptors in the immune system. Curr Opin Cell Biol. 2003; 15:539-546
33. Vivier E, Nunes J A, Vely F. Natural killer cell signaling pathways. Science. 2004; 306:1517-1519
34. Rogers N C, Slack E C, Edwards A D, Nolte M A, Schulz O, Schweighoffer E, Williams D L, Gordon S, Tybulewicz V L, Brown G D, Reis ESousaC. Syk-Dependent Cytokine Induction by Dectin-1 Reveals a Novel Pattern Recognition Pathway for C Type Lectins. Immunity. 2005; 22:507-517
35. Herre J, Marshall A S, Caron E, Edwards A D, Williams D L, Schweighoffer E, Tybulewicz V, Reis eSousaC, Gordon S, Brown G D. Dectin-1 uses novel mechanisms for yeast phagocytosis in macrophages. Blood. 2004; 104:4038-4045
36. Fallon R J, Danaher M, Saylors R L, Saxena A. Defective asialoglycoprotein receptor endocytosis mediated by tyrosine kinase inhibitors. Requirement for a tyrosine in the receptor internalization signal. J Biol Chem. 1994; 269:11011-11017
37. Geffen I, Fuhrer C, Leitinger B, Weiss M, Huggel K, Griffiths G, Spiess M. Related signals for endocytosis and basolateral sorting of the asialoglycoprotein receptor. J Biol Chem. 1993; 268:20772-20777
38. Chen M, Kakutani M, Naruko T, Ueda M, Narumiya S, Masaki T, Sawamura T. Activation-dependent surface expression of LOX-1 in human platelets. Biochem Biophys Res Commun. 2001; 282:153-158
39. Dean Y D, McGreal E P, Gasque P. Endothelial cells, megakaryoblasts, platelets and alveolar epithelial cells express abundant levels of the mouse AA4 antigen, a C-type lectin-like receptor involved in homing activities and innate immune host defense. Eur J Immunol. 2001; 31:1370-1381
40. Testi R, Pulcinelli F, Frati L, Gazzaniga P P, Santoni A. CD69 is expressed on platelets and mediates platelet activation and aggregation. J Exp Med. 1990; 172:701-707
41. Joseph M, Capron A, Ameisen J C, Capron M, Vorng H, Pancre V, Kusnierz J P, Auriault C. The receptor for IgE on blood platelets. Eur J Immunol. 1986; 16:306-312

1. A method for screening compounds to identify therapeutic candidates for treating hemostasis disorders, said method comprising:
contacting a cell that expresses a CLEC-2 receptor with a test compound and optionally, a CLEC-2 receptor ligand, under conditions which, but for the presence of the test compound, permit binding of the CLEC-2 receptor to the CLEC-2 receptor ligand; and
detecting whether the compound increases or decreases an indicator of CLEC-2 receptor activity in comparison to a control;
wherein an increase or decrease in the indicator in comparison to the control indicates that the test compound is a candidate for modulating hemostasis.

2. The method according to claim 1, wherein the indicator is selected from the group consisting of a) cell activation; b) tyrosine phosphorylation of CLEC-2; and c) interaction of CLEC-2 with a member of the Syk family of protein kinases.

3. The method according to claim 2, wherein cell activation is indicated by a change in cell shape or cellular aggregation.

4. The method according to claim 1, wherein said CLEC-2 receptor ligand is an antagonist of CLEC-2 receptor activity.

5. The method according to claim 1, wherein said CLEC-2 receptor ligand is an agonist of CLEC-2 receptor activity.

6. The method according to claim 5, wherein said CLEC-2 receptor ligand is rhodocytin.

7. The method according to claim 5, wherein said CLEC-2 receptor ligand is an antibody.

8. The method according to claim 1, wherein said cell is a platelet.

9. The method according to claim 1, wherein said decrease of said indicator indicates that said test compound is a candidate for decreasing cellular aggregation.

10. The method of claim 9, wherein said decrease of said indicator indicates that said test compound is a candidate for decreasing platelet aggregation.

11. A method for screening compounds to identify therapeutic candidates for treating hemostasis disorders comprising the steps of a) contacting a cell that expresses a CLEC-2 receptor with a test compound; and b) detecting any change in an indicator of CLEC-2 receptor activity in comparison to a control.

12. The method according to claim 11, wherein said increase of said indicator indicates that said test compound is a candidate for increasing cellular aggregation.

13. A method for screening compounds to identify therapeutic candidates for modulating hemostasis, said method comprising:
administering a test compound and a CLEC-2 receptor ligand to a non-human animal that expresses a CLEC-2 receptor; and
detecting whether said compound increases or decreases an indicator of CLEC-2 receptor activity in said animal in comparison to a control animal;
wherein a decrease in said indicator indicates that said compound is a candidate for decreasing cellular aggregation by decreasing CLEC-2 receptor activity in vivo; and
wherein an increase in said indicator indicates that said compound is a candidate for increasing cellular aggregation by increasing CLEC-2 receptor activity in vivo.

14. The method of claim 13, wherein said animal is a transgenic non-human animal which has been transformed with an exogenous nucleic acid sequence encoding a CLEC-2 receptor which is expressed in said animal, or a descendant of such an animal.

15. The method according to claim 13 wherein said cellular aggregation is platelet aggregation.

16. The method of claim 13, wherein said indicator is selected from the group consisting of cellular shape change, cellular aggregation, tyrosine phosphorylation of CLEC-2, and interaction of the CLEC-2 cytoplasmic tail with a member of the syk family of protein kinases.

17. A method for screening compounds to identify therapeutic candidates for modulating hemostasis, said method comprising:
contacting a compound and a CLEC-2 receptor polypeptide or fragment thereof; and
detecting binding between said CLEC-2 receptor binding domain and said compound.

18. The method of claim 17, wherein said CLEC-2 fragment is SEQ ID NO: 3.

19. A method for screening compounds to identify therapeutic candidates for treating homeostasis disorders, said method comprising the steps of:
contacting a CLEC-2 polypeptide or fragment thereof having a phosphorylated tyrosine in a cytoplasmic domain, with a test compound; and
detecting whether the compound modulates the binding of a signaling partner to the CLEC-2 polypeptide;
wherein said signaling partner is a member of the Syk family of tyrosine kinases.

20. The method of claim 19, wherein said member of the Syk family is Syk.

21. The method of claim 19, wherein said compound blocks the binding of said CLEC-2 to said signaling partner by selectively binding to the phosphorylated cytoplasmic domain of said CLEC-2 receptor.

22. The method of claim 19, wherein said compound blocks the binding of said CLEC-2 to said signaling partner by selectively binding to said signaling partner.

23. The method of claim 19, wherein said compound blocks the interaction between the CLEC-2 and the signaling partner and reduces cellular aggregation of a CLEC-2 expressing cell.

24. The method of claim 23, wherein said cellular aggregation is platelet aggregation.

25. A method for reducing the severity of a pathological state mediated by CLEC-2 signaling comprising the method of contacting a CLEC-2 polypeptide or fragment thereof having a phosphorylated tyrosine in a cytoplasmic domain with a compound which blocks the binding of said signaling partner to said CLEC-2.

26. A method for reducing the severity of a pathological state mediated by CLEC-2 signaling comprising the method of administering to a mammal an antagonist of CLEC-2 signaling.

27. The method of claim 26, wherein said pathological state is selected from the group consisting of thrombosis, inflammation, and acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent, cardiac valve, and conditions requiring the fitting of prosthetic devices.

28. A method for reducing the severity of a pathological state mediated by a lack of CLEC-2 signaling comprising the method of administering to a mammal an agonist of CLEC-2 signaling.

29. The method of claim 28, wherein the pathological state is excessive bleeding.

30. A CLEC-2 antagonist comprising an isolated antibody, which specifically binds to a polypeptide of SEQ ID NO: 2, or fragments thereof.

31. The CLEC-2 antagonist of claim 30, further comprising a pharmaceutical carrier.

32. The CLEC-2 antagonist of claims 30 or 31, wherein the antibody is: a) a chimeric antibody; b) a Fab fragment, c) a F(ab′)₂ fragment, or d) a humanized antibody.

33. A pharmaceutical composition comprising a polypeptide of SEQ ID NO: 2, or fragments thereof, or a salt thereof, and a pharmaceutical carrier.

34. A pharmaceutical composition comprising a nucleic acid encoding SEQ ID NO: 1, or fragments thereof or a salt thereof and a pharmaceutical carrier.


SEQUENCE LISTING (16 sequences)

SEQ ID NO: 1 - DNA, 820 bp, Artificial Sequence
  Description of Artificial Sequence Nucleotide sequence corresponding to the human CLEC-2 receptor
aaactacattttgcaaagtcattgaactctgagctcagttgcagtactcgggaagccatgcaggatgaagatggatacatcaccttaaatattaaaactcggaaaccagctctcgtctccgttggctctgcatcctcctcctggtggcgtgtgatggctttgattctgctgatcctgtacgtggggatggttgtcgggctggtggctctggggatttggtctgtcatgcagcgcaattacctacaagatgagaatgaaaatcgcacaggaactctgcaacaattagcaaagcgcttctgtcaatatgtggtaaaacaatcagaactaaagggcactttcaaaggtcataaatgcagcccctgtgacacaaactggagatattatggagatagctgctatgggttcttcaggcacaacttaacatgggaagagagtaagcagtactgcactgacatgaatgctactctcctgaagattgacaaccggaacattgtggagtacatcaaagccaggactcatttaattcgttgggtcggattatctcgccagaagtcgaatgaggtctggaagtgggaggatggctcggttatctcagaaaatatgtttgagtttttggaagatggaaaaggaaatatgaattgtgcttattttcataatgggaaaatgcaccctaccttctgtgagaacaaacattatttaatgtgtgagaggaaggctggcatgaccaaggtggaccaactaccttaatgcaaagaggtggacaggataacacagataagggctttattgtacaataaaagatatgtatgaatgcaaaaaa

SEQ ID NO: 2 - protein, 229 aa, Artificial Sequence
  Description of Artificial Sequence Peptide sequence corresponding to the human CLEC-2 receptor
MQDEDGYITLNIKTRKPALVSVGSASSSWWRVMALILLILYVGMVVGLVALGIWSVMQRNYLQDENENRTGTLQQLAKRFCQYVVKQSELKGTFKGHKCSPCDTNWRYYGDSCYGFFRHNLTWEESKQYCTDMNATLLKIDNRNIVEYIKARTHLIRWVGLSRQKSNEVWKWEDGSVISENMFEFLEDGKGNMNCAYFHNGKMHPTFCENKHYLMCERKAGMTKVDQLP

SEQ ID NO: 3 - protein, 13 aa, Artificial Sequence
  Description of Artificial Sequence Peptide sequence to identify human CLEC-2-binding molecules
MQDEDGYITLNIK

SEQ ID NO: 4 - DNA, 39 bp, Artificial Sequence
  Description of Artificial Sequence Oligonucleotide sequence to identify human CLEC-2-binding molecules
atgcaggatgaagatggatacatcaccttaaatattaaa

SEQ ID NO: 5 - protein, 5 aa, Artificial Sequence
  Description of Artificial Sequence Synthetic peptide
GRGDS

SEQ ID NO: 6 - protein, 6 aa, Artificial Sequence
  Description of Artificial Sequence Synthetic peptide (thrombinreceptor agonist peptide, TRAP)
SFLLRN

SEQ ID NO: 7 - protein, 13 aa, Artificial Sequence
  Description of Artificial Sequence Synthetic peptide
MQDEDGYITLNIK

SEQ ID NO: 8 - protein, 9 aa, Homo sapiens
TGTLQQLAK

SEQ ID NO: 9 - protein, 11 aa, Homo sapiens
YYGDSCYGFFR

SEQ ID NO: 10 - protein, 5 aa, Homo sapiens
THLIR

SEQ ID NO: 11 - protein, 10 aa, Homo sapiens
NYLQDENENR

SEQ ID NO: 12 - protein, 8 aa, Homo sapiens
RFCQYVVK

SEQ ID NO: 13 - protein, 7 aa, Homo sapiens
FCQYVVK

SEQ ID NO: 14 - protein, 7 aa, Homo sapiens
HYLMCER

SEQ ID NO: 15 - protein, 9 aa, Homo sapiens
MHPTFCENK

SEQ ID NO: 16 - protein, 4 aa, Artificial Sequence
  Synthetic peptide
YXXL